(19) United States
(12) Patent Application Publication
(10) Pub. No.: US 2010/0061892 A1
(43) Pub. Date: Mar. 11, 2010

(54) MICROFLUIDIC DEVICE HAVING AN ARRAY OF SPOTS

(75) Inventors: Eric Flaim, Edmonton (CA); Daniel J. Harrison, Edmonton (CA); Mark T. McDermott, Edmonton (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: THE GOVERNORS OF THE UNIVERSITY OF ALBERTA, Edmonton, AB (CA)

(21) Appl. No.: 12/513,347

(22) PCT Filed: Nov. 5, 2007
(86) PCT No.: PCT/CA07/01984

§ 371 Date: May 20, 2009

Related U.S. Application Data
(60) Provisional application No. 60/864,214, filed on Nov. 3, 2006.

(51) Int. Cl. G01N 33/00

(52) U.S. Cl. 422/68.1

(57) ABSTRACT

A microfluidic spotting device has a first substrate patterned with an array of spots, a second substrate attached directly or indirectly to the first substrate, and channels formed at least partly in at least one of the first substrate and the second substrate, each channel having an inlet channel and an outlet channel.

### BACKGROUND

Analytical techniques for use in biomedical applications have developed requirements for simultaneous multiple sample sensing analytical devices. As an example, Surface Plasmon Resonance (SPR) has emerged as a powerful bio-analytical tool for both research and clinical applications, particularly because it does not require labeling of the analyte. SPR is an optical technique capable of detecting non labeled analytes at coinage metal, such as gold (Au) and silver (Ag), thin films by measuring changes in refractive index upon binding of analytes to the sensor surface.

The SPRI (Surface Plasmon Resonance Imaging) sensor chips that have been developed with patterned areas of gold provide high detection contrast, but suffer difficulties such as requiring robotic pin printing, manual pipetting techniques, and surface chemistry modifications.

### SUMMARY

There is provided in one embodiment a microfluidic spotting device, comprising a substrate patterned with an array of spots, as for example metal spots; a channeled substrate attached to the substrate; and a channel network formed between the spotted substrate and the channeled substrate, each spot being in communication with a channel path through the channel network. The channel network may comprise channels formed at least partly in at least one of the first substrate and the second substrate, each spot being in communication with an inlet channel leading to the spot and an outlet channel leading away from the spot.

Various embodiments of the microfluidic spotting device may have one or more of the following features:
1. the substrate is suitable for surface Plasmon resonance analysis;
2. each channel path, comprising an inlet channel and outlet channel, is uniquely associated with and passes across a spot or group of spots;
3. each channel path has a length, the lengths of the channel path are equal and each channel presents equal resistance to flow through the channels;
4. at least one spot in a channel is an elongate spot extending along the channel;
5. at least one spot is formed as part of a contiguous strip passing across multiple channels;
6. at least one channel has more than one inlet channel;
7. more than one outlet channel is connected to a common drain;
8. at least one inlet channel is in communication with a reaction bed upstream from the corresponding spot;
9. a top substrate is attached to the second substrate such that the second substrate is an intervening substrate between the top substrate and the first substrate;
10. one or more intervening substrates have openings corresponding to the locations of spots on the spotted substrate;
11. at least one channel is at least partially formed in a top substrate;
12. the second substrate is attached directly or indirectly to the spotted substrate by an attachment surface, the channel network being formed on the attachment surface of the second substrate;
13. the array of spots is an array of coinage metal spots;
14. the channel network comprises channels, and the channels are parallel to the plane of the array of spots; and
15. the inlet channels of the channel network are connected to receive fluid from a microtitre plate.

In another embodiment, there is provided a method of operation of a microfluidic spotting device, in which spots patterned on a substrate are supplied analyte from corresponding wells of a microtitre plate.

In another embodiment, there is provided a method of manufacturing a microfluidic spotting device in which spots are patterned in an array on a base substrate, followed by attachment, directly or with an intervening spacer, of a channeled substrate to the base substrate, in which channels of the channeled substrate provide inlet channels and outlet channels for the spots in the array.

In another embodiment, there is provided a method of providing a mask, for example for creating an array of spots in a pattern on a substrate, comprising forming a positive relief corresponding to the pattern, applying a moldable material to the positive relief, setting the moldable material and removing the moldable material from the positive relief.

In another embodiment, there is provided a method of patterning spots on a substrate comprising creating a mask having windows corresponding to a desired array of spots and exposing a substrate to a vapour flux through the mask.

In another embodiment, there is provided a simple micro scale gold patterning technique for use with a unique microfluidic spotting device to create a convenient and customizable microarray platform for Surface Plasmon Resonance Imaging.

### BRIEF DESCRIPTION OF THE FIGURES

Embodiments will now be described with reference to the figures, in which like reference characters denote like elements, by way of example, and in which:

FIG. 1A through 1F is a schematic representation of the PDMS shadow mask fabrication.

FIG. 2 is a schematic view of a 24 spot microfluidic device and its channel network.

FIG. 3 is a detailed top plan view of spotting regions.

FIG. 4 is a side elevation view in section of a fully aligned 96 spot device.

FIG. 5 is an image of a 24 spot array.

FIG. 6 is a detailed top view of a spotting substrate coupled with two PDMS substrates.

FIG. 7 is a detailed side view in section of a spotting substrate coupled with two PDMS substrates.

FIG. 8 is a detailed side view in section along the channel of a spotting substrate coupled with two PDMS substrates.

FIG. 9 is a schematic view of a channel having a digestion bed and multiple spotting regions.

FIG. 10 is a schematic view of a channel having a preconcentration bed for each spotting region.

FIG. 11 is a schematic view of a mixing channel with multiple inlets.

FIG. 12 is a perspective view of a simplified microfluidic spotting device.

FIG. 13 is a side view in section of the microfluidic spotting device of FIG. 12.

FIG. 14 is a detailed perspective view of a simplified microfluidic spotting device (not to scale) with an intervening substrate.

FIG. 15 is a schematic view of a 20-spot microfluidic device and its channel network.

FIG. 16 is a schematic view of a channel network with elongate spots.

FIG. 17 is a schematic view of a channel network with multiple spots per channel.

FIG. 18 is a schematic view of a channel network with channels perpendicular to strips.

### DETAILED DESCRIPTION

### Fabrication of a Microfluidic Spotting Device

The device described herein allows for gold patterning to achieve high viewing contrast and can accommodate various solution types without surface modifications. In addition, it may limit the effect of evaporative loss, which results in sample drying and denaturation that occurs with high surface area to volume ratios. The device is therefore useful, for example, in low density sample requirements that do not justify the burdening cost of high through put systems and their time consuming protocols, such as labeling.

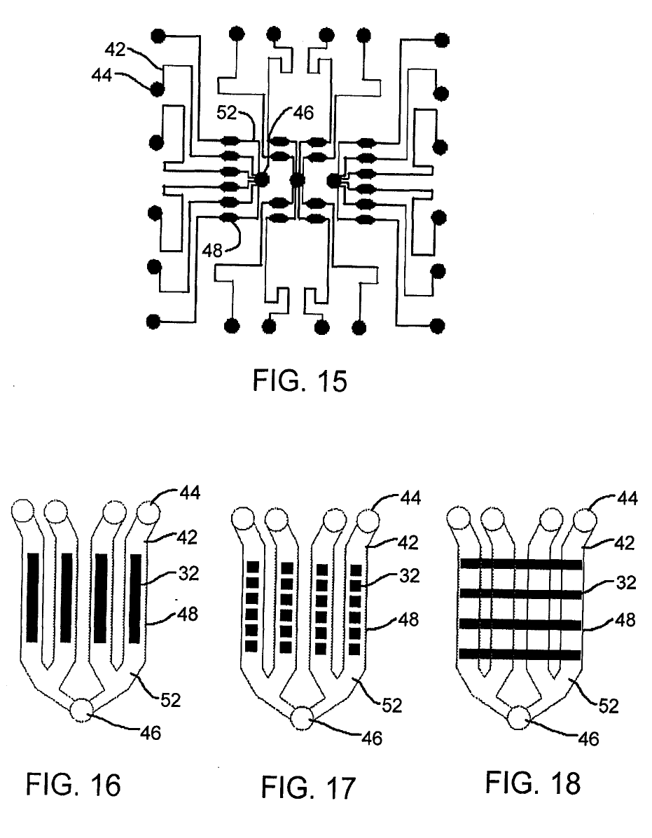

Referring to FIG. 12, a microfluidic spotting device 10 has a first substrate 16 patterned with spots 32 of material that can be used for detection purposes. For example, coinage metal is commonly used in SPR techniques. A second substrate 34 is attached to the first substrate 16. This attachment may be made directly or indirectly, as for example through an intervening layer. Channels 42, 50 and 52 of a channel network are formed by attaching the substrates 16 and 34 together. This may be done by forming each channel in either the first substrate 16, the second substrate 34, or partly in each, or in nor partly in an intervening layer. In one embodiment, each spot is in communication with a distinct channel path through the channel network that is uniquely associated with the spot. That is, for each spot, there is one and only one channel path for the spot. Each channel 42 forms an inlet channel leading to a spot 32, while for each spot 32 there is an outlet channel 52. The outlet channels 52 may be combined into a single outlet channel 50, or may terminate in a common sink or drain, as for example drain 46 in FIG. 15.

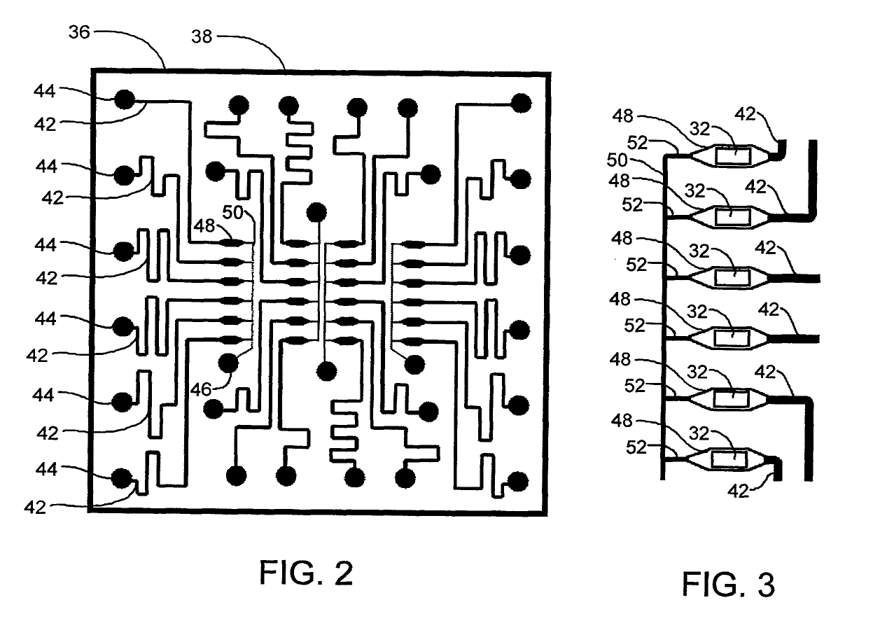

Referring to FIGS. 2 and 3, examples of spotting devices 10 are shown. Each spot 32 is patterned on a substrate. A channel network is formed in an overlying substrate. Within the channel network, there is a spotting region 48 corresponding to each spot 32. Each channel path passing across a spot 32 through a spotting region 48 has an inlet channel 42 leading to the spot 32, and an outlet channel 52 leading away from the spot 32. As shown in FIG. 2, multiple outlet channels 52 converge into a single drain channel 50 leading to a drain outlet 46. In use a vacuum is applied to the drain outlets 46 to draw fluids through the inlet channels 42 to come into contact with the spots 32. The example shown in FIG. 12 uses a shared outlet channel 52 for two spots 32. Different channel arrangements may be used, depending on the intended application. The arrangement may range from very simple to very complex.

Another example of a channel network for a microfluidic device is shown in FIG. 15. In this embodiment, the outlet channels 52 meet at the common drain outlet 46 rather than a common outlet channel, as in FIG. 2. Fluid inlet channels 42 have been designed such that the length of each inlet channel associated with a drain outlet 46 is the same length, and that the cross-section of each inlet and outlet channel 42 and 52 is the same. The length of a channel is the distance between an inlet reservoir and a drain reservoir. By not sharing a common outlet channel, but sharing a common drain, equal resistance to flow in each channel can be achieved. A desired volume flow rate for a given applied pressure can then be controlled through the channel dimensions of length, depth and width.

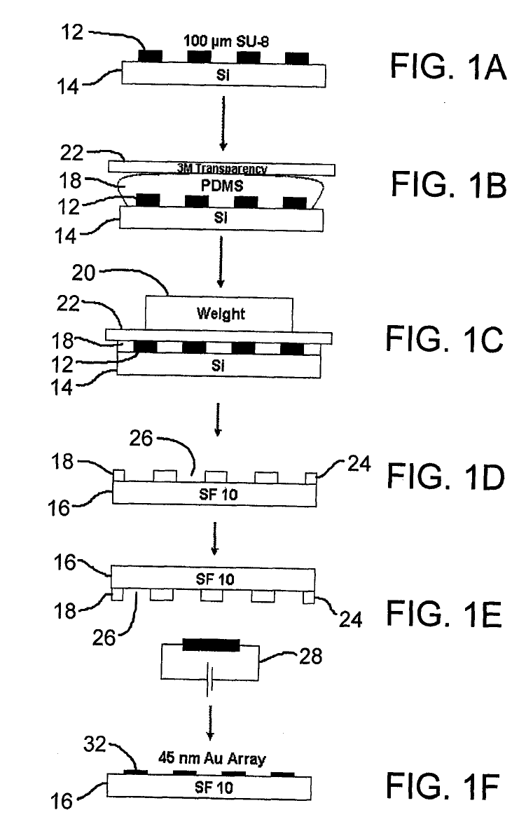

Referring to FIG. 1A through 1F, a method of patterning spots onto a substrate is shown. It will be understood that other techniques of patterning spots of desired material onto a substrate in a desired pattern may be used in some embodiments. The method that is depicted involves the photolithographic fabrication of arrays of photoresist columns corresponding to the desired spot size on a substrate. These positive relief photoresist column arrays serve as reusable masters for the formation of thin shadow mask membranes containing through holes. For example, the thin shadow mask membrane may be formed from curing PDMS around the features. If PDMS is used, a minimum height of 100 μm is generally needed for easy manual handling of a PDMS shadow mask with tweezers. Referring to FIG. 1A, photoresist 12 is cured on a masking substrate such as a silicon wafer 14, and the excess photoresist (not shown) is removed to form columns of cured photoresist 12. The photoresist pattern is made to correspond with the desired spot pattern. Referring to FIG. 1B, PDMS liquid polymer 18 is applied to the Si (silicon) wafer 14 to sufficiently cover the cured photoresist 12. To avoid curing of PDMS 18 over the features, and thus enable metal to be deposited on the glass substrate 16 shown in FIG. 1D, weights 20 may be applied to remove excess PDMS 18 from above the features formed from cured photoresist 12. A sheet 22 is used to separate the PDMS liquid polymer 18 from the weights 20 that exhibit less adhesion to the PDMS 18 compared with the adhesion of the PDMS 18 to the Si wafer 14. A transparency sheet from 3M™ may be used. Referring to FIG. 1D, upon curing, PDMS shadow mask membranes 24 with arrays of through holes 26 are removed and can be used in creating spot patterns. These mask membranes 24 may vary in size, depending on the desired size of the spotted substrate 16. In one example, mask membranes 24 that were approximately 1.8 cm² in size were cut from the bulk PDMS membrane sheet and applied to 1.8 cm² SPR glass slides 16. Once cured, the thin PDMS mask membrane 24 is transferred from the masking substrate 14 to the substrate 16 to be spotted, such as a glass slide. If PDMS and glass is used, it has been determined that the native conformal contact between the PDMS and the glass 16 provides a versatile seal allowing for localized metal deposition to the exposed areas under the through holes 26. This contact is reversible, which allows the PDMS shadow masks 24 to be reused for further metal depositions. Referring to FIG. 1E, metal 30 is then deposited onto the PDMS membranes 24 and into holes 26 to form the metal spots 32 on the substrate 16 as shown in FIG. 1F. This may conveniently be done using a thermal evaporator 28 as shown. A general layout of the resulting metal deposition may include a 4×6 array of spots as shown in FIG. 5, an 8×12 array, or other array, as desired. It will be understood that the array of spots 32 including the size and number of spots may be varied according to the intended application. For example, the device may be coupled with more conventional sample handling systems, such as microtitre plates and multichannel pipettes for the use with standard bio assay protocols. To correspond to a microtitre device (described below), a pattern having 96 spots 32 may be used. The basic steps of FIGS. 1A-1F may be used for selective patterning to a substrate for a wide variety of materials in addition to metal, such as oxides, nitrides, silanes and thiols.

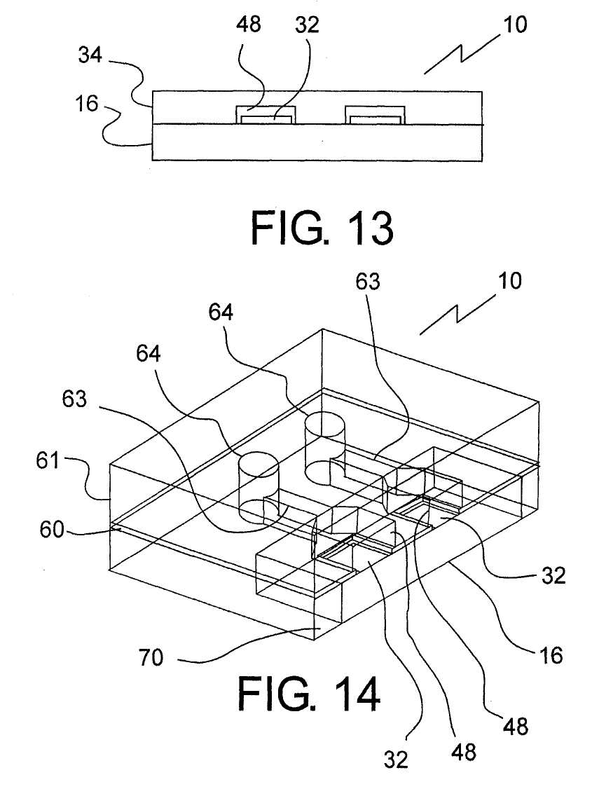

Referring to FIGS. 12 and 13, a microfluidic device 10 is formed by overlaying the pattern of spots 32 with a channeled substrate 34. For example, channeled substrate 34 may be formed of PDMS, with a spotted substrate 16 of glass. However, the channeled substrate 34 may also be fabricated using hard materials, such as glass, quartz, ceramics, neoprene, Teflon and silicon as well as a range of soft materials, such as polymer systems based on acrylamide, acrylate, methacrylate, esters, olefins, ethylene, propylene and styrene. Also, combinations of hard and soft materials allow for fabrication of the outlined devices. Fabrication of positive relief masters includes both dry and wet etching processes of hard materials. Polymer mold fabrication of these positive relief masters can be accomplished by casting, injection molding and hot embossing. Based on existing techniques, it will be understood by those in the art how to apply and/or modify the fabrication steps described below based on the type of material.

Referring to FIG. 2, the design of a master 36 used to create an exemplary channeled substrate 34 for a 24 spot microfluidic device is shown. If the channeled substrate 34 is to be formed of PDMS, master mask 36 is a positive relief photoresist master formed using standard photoresist techniques on a substrate 38, such as a silicon wafer. Multiple masters, such as four, may be formed on a single mask substrate. In one embodiment, the master 36 had a perimeter of 1.8 cm² with 100 μm wide flow channels 42, and feature heights of 40 μm.

Referring to FIG. 2, the master 36 has been designed with four specific characteristics. For convenience, similar reference numerals have been given to the positive relief elements and the corresponding elements in the channeled substrate. First, every six inlets 44 have a common outlet 46, which reduces the number of access holes needed. Second, inlet channels 42 are lengthened for extra flow restriction to ensure that the solution containing the analyte arrive at each spot at the same time. Third, referring to FIG. 3, the design allows the analyte solution to flow through a spotting region 48 to allow for complete solution coverage of the larger spots that it is designed to cover. Fourth, the outlet paths 50 of each spotting region 48 are removed from the outlet channel 52 to limit the possibility of backflow of the waste line 50 to the spotting regions 48. In one embodiment, the outlet channels 52 were 50 μm wide and removed by 300 μm.

If PDMS is to be used, after photolithography, the Si wafer 38 is silanized and PDMS 54 is cured over the master 36, such as to a height of 2 mm. If more than one master 36 is included on the channeled mask substrate 38, each channeled substrate 34 is cut from the bulk PDMS 54 and access holes 44 and 46 are made through the PDMS 54. If a diameter of 1 mm is desired, access holes 44 and 46 may be produced by using a 16 gauge needle whose tip has been flattened and sharpened to produce access holes 44 and 46. Referring to FIG. 3, the channeled substrate 34 is then aligned with the spotted glass substrate 16 using an alignment microscope (not shown) to form the microfluidic device 10, such that spots 32 are completely covered by spotting region 48. Using the dimensions from the above example, the channeled substrate 34 and spotted substrate 16 are both 1.8 cm² and can be sealed with native conformal contact. The conformal attachment between the PDMS layer 34 and glass substrate 16 proves to be a stronger attachment than on a fully coated Au slide with no leakage of aqueous or organic solutions. However, it will be understood that if an adequate attachment could be made, a fully coated substrate rather than a spotted substrate could also be used.

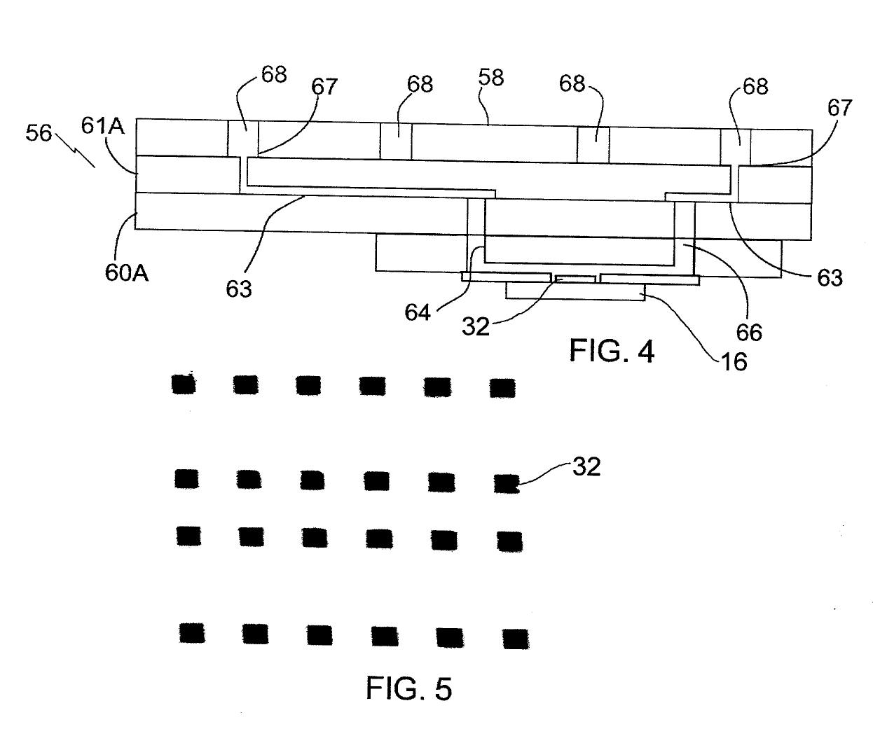

The example used to illustrate the method described above referred specifically to a 24 spot device. Many of the same fabrication techniques and features used in the 24 spot microfluidic device can be applied to a larger 96 spot/48 sample device 10. One outlet for every six inlets, elongated path lengths for fluid restriction, spot-patterned slides and spotting regions are all aspects shared in common with the 24 spot design. FIG. 4 shows a completed device 56 in section aligned and mounted to a microfluidic device 10 patterned with spots. The device is coupled to a conventional microtitre plate 58.

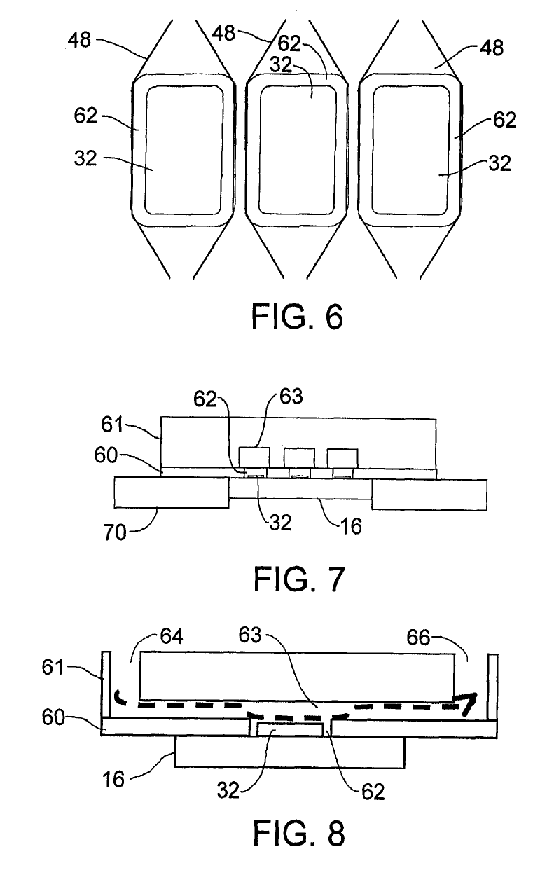

Referring to FIGS. 7 and 8, a thin intervening substrate 60 with through holes 62 has been illustrated. Referring to FIG. 14, the intervening substrate 60, which may also be formed of PDMS, is positioned between spotted substrate 16 and channeled substrate 32, creating an indirect coupling between the two substrates. The intervening substrate 60 is used in certain circumstances, such as to allow for fluid flow to be brought to the localized spots 32 from outside the 1.8 cm² SPR sensor 10, and therefore allowing for increased number of inlets 44 and outlets 46. The intervening substrate 60 also allows for the possibility of coupling to a microtitre plate 58 as shown in FIG. 4. Referring to FIG. 4, this intervening substrate 60 is irreversibly bonded to a 2 mm thick PDMS channeled substrate 61 containing negative relief channels 63. Channeled substrate 61 is formed using a similar technique to the channeled substrate formed for the spotted substrate with 24 spots described above. Fluid flow then travels along the thin intervening substrate 60, guided by channels 63, to the spotting regions 48 for deposition to the spots 32. Referring to FIG. 6, to ensure proper fluid coverage of the spots 32, with out trapping air, the access wells created by placing holes 62 in the thin intervening substrate 60 over the spotted substrate 16 lacked 90 degree angles at the corners, and were fabricated 50 μm wider on each side compared to the spots 32. Referring to FIG. 7, spotted glass substrate 16 is held by an aluminum plate holder 70. This view also shows the relation between channels 63, access wells 62, and spotted substrate 16. The channels 63 typically extend for some distance across the substrate as shown in FIG. 4.

Referring to FIG. 4, inlet ports 64 and outlet ports 66 are formed in the channeled substrate 61 by punching through the cured PDMS, such as with a hollowed 3 mm ID steel rod with a sharpened tip. To couple to the microtitre plate 58, holes 67 are drilled through the wells 68 of the microtitre plate 58. It is preferred that holes 67 are smaller in diameter than the inlet ports 64 and outlet ports 64, such as 2 mm. Thus, since the microtitre plate wells 68 are conical in shape, they sit flat within the larger wells of the access holes 64 in the channeled substrate 61. Transport of the solution containing the analyte through the channels of the device to and from the spotting regions may be achieved by applying vacuum to the outlets, by applying pressure to the inlets, or by using electrokinetic forces.

The fabrication steps described above can be used to help develop a simple microscale patterning technique for use with a unique microfluidic spotting device to create a convenient and customizable microarray platform for techniques such as Surface Plasmon Resonance Imaging. It has been found that using a pattern of spots is beneficial in performing multi-analyte analysis in a microarray format. For example, surface plasmon resonance (SPR) only occurs at the surfaces of coinage metals when certain conditions of wavelength and angle are met. Thus, to localize the SPR response and minimize the background signal that is generated across the whole surface of an SPR sensor chip, patterning of Au spots may be used. The size of the spot to be patterned will depend upon the ease of visualization with the detection equipment, such as an SPR Imager for SPR, and the microfluidic solution delivery system that it must be coupled to. For the SPR results discussed below, sufficient results were achieved by using an exemplary spot size of 500×300 μm². As an example, photolithographic techniques can be used to create spot patterns of such size. It will be understood that the limit to spotting density is affected more by design requirements and the size of sensing surfaces than by the fabrication process. Smaller spots, and accompanying channels in channeled substrate (described below), can be made, thereby increasing spot density to be compatible with the resolution achievable with a microscopy detection system such as reflection IR and fluorescence microscopy.

Photoresist lift off is one technique used for metal patterning on substrates of glass, and in particular for SPR, patterning gold and silver. Specific patterning of hard materials and reactive compounds, with functionalized end groups, can be achieved. Photoresist lift off uses photolithography to pattern photoresist on the substrate of interest. Upon UV exposure and development, metals can be deposited on the underlying substrate. Once metal deposition is completed the remaining photoresist can be removed leaving behind the patterned metal. However, the process below was used in an attempt to simplify the procedure and eliminate possible surface contamination of the substrate and metal from the photoresist removal.

Reflection IR and fluorescence microscopy do not require the same spot size as does SPR. Therefore, to maintain a two layer device within approximately the same substrate dimensions, it would be possible to increase the number of spots, such as from 96 to 192 using dimensions given above. Further increases, for example to 384, can be accomplished by adding additional layers for added flow channels. The channels are formed using steps similar to those above, with the channels in one layer being sealed as they are coupled to the adjacent layer. Appropriately positioned holes then allow the fluid to flow downward through each layer to reach the spotting region on the glass substrate. This allows fluid passage to a specific region on the substrate, and an increased channel density. This also allows for greater flexibility when compared with a single layer having a micro trench placed in a face-to-face orientation against a substrate. Stacking of layers, and passage of fluids from one layer to another through access wells is only limited by the spot density desired for a substrate of a given area. In addition, connection tubing may connect directly to the inlets and outlets. In this embodiment, the device may then be incorporated directly into a detection device, such that analyte could be continuously supplied to the spotting regions during detection.

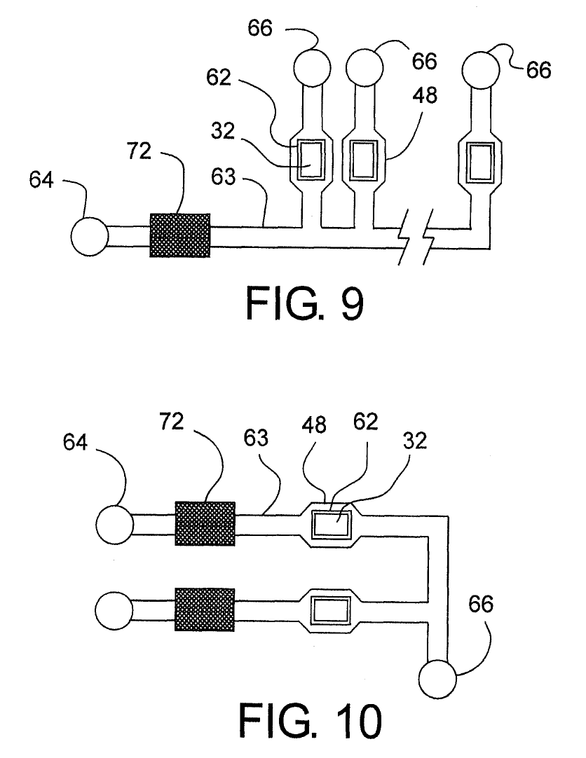

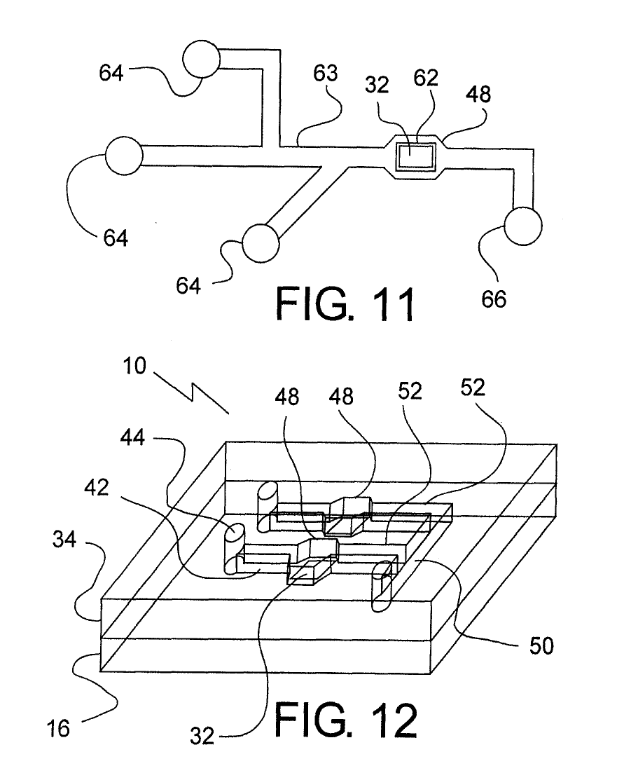

The microfluidic device 10 is not limited to inlets, delivery channels, spotting regions and outlets as described to this point. More sample preparation steps may be integrated into the device. For example, referring to FIG. 10, a reaction bed 72, such as a preconcentration bed, also referred to as a solid phase extraction bed, may be included before the spotting region 48 to concentrate samples. Referring to FIG. 9, the reaction bed 72, such as a digestion or enzymatic bed, may be placed at a common inlet 64 for fractionation of reaction products to individual spotting regions 48. Referring to FIG. 11, multiple inlets 64 may be connected to a single spotting region 48 to allow the user to mix samples prior to spotting. Referring to FIG. 8, reaction bed 72 may be filled with polymer material in the manner known to those who make monolithic structures. Generally, monolithic structures are formed by filling an untreated capillary with a polymerization mixture, and initiating the radical polymerization thermally using an external heated bath. Once the polymerization is complete, the unreacted components are removed from the monolith. A weir may be provided around the reaction bed 72 to trap the packing material within it. Other channels (not shown) than those intended for the solution carrying the analyte may be used to deliver the material to the reaction bed.

Referring to FIG. 16, the spotting regions 48 of the channel network may be designed to accommodate elongated spots 32 in the form of strips of material. When mounted into an SPR detection system, samples may be flowed through the channels for real time SPR detection. In this way the device can be used as a sample flow cell for SPR detection on the patterned array. This allows for simultaneous investigation of different samples along with a minimization of sample volume. Alternatively, referring to FIG. 17, the spotting regions 48 may accommodate multiple spots 32 per channel. This increases the number of reaction sites per channel. Another way of achieving multiple spots per spotting region 48 is to place the channels perpendicular to spots 32 formed of contiguous metal strips, as shown in FIG. 18. The length of the inlet channels 42 corresponding to each spotting region 48 is the same, and the channels each present equal flow resistance, and that the outlet channels 52 all connect to a single outlet drain 46.

The fabrication methods described above may be used to create a microfluidic device 10 that may then be used for patterning chemicals of interest for any surface based analysis method, such as ellipsometry, Surface Plasmon Resonance (SPR) Imaging, infrared and fluorescence spectroscopy, etc. Microfluidic device 10 is not limited to the application of label free microarrays utilizing Surface Plasmon Resonance Imaging (SPRI) detection that is described below.

### Demonstrations of Capability In SPR Imaging

There will now be given a description of the use of microfluidic device 10 in Surface Plasmon Resonance Imaging (SPRI), in which it acts as a label free microarray. SPR is an optical technique capable of detecting non labeled analytes at coinage metal (Au, Ag) thin films by measuring changes in refractive index upon binding of analytes to the sensor surface. SPR Imaging (SPRI) maintains a constant viewing angle where differences due to adsorption events can be recorded as differences in reflectivity intensities over the entire sensor surface. SPRI has emerged as a convenient method for multi-analyte analysis in a microarray format and has been applied to peptide protein, protein protein and carbohydrate protein binding events. To be used for SPRI, the present device is designed to combine gold patterning to achieve high viewing contrast, to allow for various solution types, and to limit the effect of drying and denaturation that occurs with high surface area to volume ratios. The present device uses a SPR-inert substrate, meaning that the substrate doesn't give off any emissions or signals during SPRI. A convenient material to use for this is glass, although other materials may also be used. In addition, since SPRI can be performed with the PDMS layer on top, it avoids any contamination or drying that may otherwise occur.

Typical SPRI sensing is accomplished on fully coated glass slides. However, to ensure no sensing complications arise from gold patterned slides, Au spotted SPR slides 14, with arrays of 4×6 and 12×8, were mounted in the SPR to observe their localized signals. SPR images of 24 and 96 spot sensors were taken with unmodified Au spots in a background solution of water. The angle was adjusted to the SPR angle resulting in minimum reflectivity of the Au spots. The remaining, uncoated-glass, background exhibited no surface plasmons due to the absence of the gold which, results in maximum reflectance of the incoming light. Thus, areas of interest were clearly visible without the need for background blocking.

The SPR images showed well defined boundaries of the Au spots 32, which was an indication of the effectiveness of the PDMS masking layers used during metal deposition (as described with respect to FIG. 1A through 1F above) to produce well defined spots across a large surface area. Such fidelity of metal deposition results in even SPR signal strength across the array with no spatial dependence. These well defined areas also exhibited no shadowing effect due to the angled path of the incoming and reflecting light.

### Organic Solution Immobilization

Gold coated substrates have been used extensively due to their ease in surface modification with alkyl thiols. Thiol adsorption to gold is thought to occur through the formation of a gold sulfur co-ordinated covalent bond, which allows for the controlled modification of the surface to many different types of chemistries through various functionalized alkyl thiols. Many investigations have occurred examining the protein binding capabilities of various functionalities for both anti fouling and high adsorption binding surface modifications. Alkyl thiols of interest are used in an ethanol solvent due to the polar nature of the alkyl chain connecting the thiol on one end and the functional group of interest on the other. Ethanol solutions are difficult to spot immobilize due to their high rate of evaporation and tendency to spread on non-polar surfaces. Reports investigating various alkyl thiol functionalities therefore modify the surface of an entire sensor using a large volume of solution, requiring individual experiments for each surface modification.

In one experiment, a 24 spot device was used to simultaneously immobilize 4 different alkyl thiols dissolved in 100% ethanol. Undodecal alkyl thiols with —NH₂, —COOH, —OH and —CH₃ functional groups were flowed through the PDMS microfluidic channels and allowed to immobilize for 2 hours at a concentration of 2 mM. Due to the small exposed surface area to volume ratio of the ethanol solutions within the microchannels there was limited solution evaporation on the time scale of immobilization. The ethanol solutions were removed by vacuum applied to the outlets of each row of six spots, and the PDMS microchannel device was removed. After an ethanol rinse and N₂ drying of the SPR slide, the slide was mounted into the SPR. It will be understood that, if the entire device were mounted into the SPR itself, it would not be necessary to remove the PDMS. This feature allows the device to be incorporated into different detection systems and to be used directly with connection tubing at the inlet and outlets to introduce and remove samples while investigating real time binding events in each spotting region.

A solution of 430 nM human fibrinogen (Hf) was then flushed through the SPR and the subsequent signal was observed for each type of functionalized surface. Based on the difference image collected upon non-specific physical adsorption of Hf to the various surface chemistries, their approximate percent reflectivities were found to be: —NH₂=43%, —OH=7%, —CH₃=27%, and —COOH=22%. The trends observed for adsorption correspond to that reported in literature for the binding of fibronectin. Greater adsorption of Hf occurs to the—NH₂ terminated thiol surface which has been reported as the most suitable for nonspecific physical adsorption. The least adsorption is observed for the alcohol terminated thiol chain which is often used for their anti fouling abilities.

### Specific Addressing

A fully customizable microarray device must allow for single spot addressability as a means for increased sample density and flexibility. In the examples given below, the 24 spot and 96 spot devices are used for direct immobilization of different proteins to various spots within the microchannel devices. Upon immobilization of various proteins, their antibodies can be flowed over the sensor surface within the SPR, to monitor specific binding of the antibody antigen pair. Where there is binding between the injected antibody and the surface immobilized antigen there is an increased SPR signal, reported with increased reflectivity. Using SPR difference images of antibody antigen binding for both a 24 and 96 spot device, it was found that the approximate percent reflectivity for each adsorbed protein was, for the 24 spot device: human fibrogen=42% and BSA=2%, and for the 96 spot device, human fibrogen=16.5%, and bovine IgG=1.5%.

A difference image was taken of 667 nM human IgG and 0.01% BSA immobilized on the Au spots in the 96 spot device. They were absorbed to the surface for one hour followed by 10 min. incubation in the SPR with 133 nM of anti-human IgG. The difference image showed the specific binding between the anti-human IgG and human IgG, with little non specific binding to the immobilized BSA, used often as a blocking agent. The human IgG has been addressed to spots, forming the letters UA. In the same way, human fibrinogen and bovine IgG were immobilized with the 96 spot device at concentrations of 470 nM and 667 nM, respectively. They were incubated with 133 nM nM anti-human fibrinogen resulting in a difference image of quadrants. In both cases, the addressable spots showed reproducible signal strength.

Low density microfluidic spotting devices for label free protein microarrays may thus be designed using micro scale metal deposition techniques coupled with a microchannel design. For example, the use of thin membrane masking layers, as for example PDMS, for metal deposition can be further extended to create larger arrays of patterned metals with any desired dimension, only limited by the master wafers aspect ratios. For use with SPR, this technique resulted in high contrast images with zero background, due to the absence of gold, and well defined, reproducible, sensing regions of interest.

Using the principles herein, a device can be made that allows for immobilization of aqueous and organic solutions within a microenvironment that does not tend to lead to evaporation or leakage. In the case of the exemplary PDMS design, microchannels are either in conformal contact with a glass slide, as in the case of the 24 spot device, or irreversibly bonded to a thin PDMS sheet, as in the case of the 96 spot device, strong seals are formed and maintained. This design permits multiple organic samples to be immobilized and investigated simultaneously within one experiment. This may be advantageous in limiting experiments when searching for the optimal gold surface modification for different protein immobilization schemes.

Specific addressing of spots is achievable with these devices allowing for complete customizability of surface immobilization. Use of such a device allows researchers to investigate their own molecules of interest adsorbed to the surface for probing with different targets. Clinical and laboratory research applications often require low density assay procedures as only few rare samples will be tested. Thus, a high through put system requiring large amounts of sample is impractical. By coupling the larger 96 spot device to familiar microtitre plates or having them align to standard multichannel pipettes, protocols for assay investigations may be co-opted to this new investigative or diagnostic platform.

### EXPERIMENTAL EXAMPLE

### Chemicals

All proteins used were purchased in the highest available purity from Sigma Aldrich and used as received. All antigen proteins were dissolved in (0.02M phosphate, 0.150M NaCl) phosphate buffered saline pH=7.4 from which they were aliquoted to their appropriate concentrations determined from the measured weight and accurate molecular mass. Antibody concentrations were determined by the dilution, with PBS, of the received commercial antisera.

Mercaptoundecylamine hydrochloride was obtained from Dojindo Laboratories (Japan); 11-Mercaptoundecanoic, 11-Undecanethiol, 11-Mercapto-1-undecanol were all purchased from Sigma Aldrich.

### Surface Plasmon Resonance Imaging

Arrays were imaged using GWC Instruments SPRimager II (GWC Instruments; Madison, Wis.) and has been described in detail elsewhere. Referring to FIG. 1A through 1F, the array sensor is constructed from the thermal evaporation of a 45 nm gold film deposited on SF10 glass (Schott; Toronto, ON, Canada) with a 1 nm adhesive chromium layer. The sensor is mounted within a fluid cell to which solutions are introduced to the entire surface via a peristaltic pump. The SPR angle is determined and then maintained during the entire course of the experiment. Images are generated from the averaging of 30 individual pictures.

Difference images are determined by subtracting the image taken after a binding event from a reference image taken prior to the binding event. Since the SPR angle is maintained any differences between the images, as a result of binding from the incubation solution, appear as illuminated areas. The value of Δ% R, is obtained, as specified by the manufacturer, by Δ% R=(0.85Iₚ/Iₛ)·100% where Iₚ and Iₛ are the reflected light intensities detected using p and s polarized light.

### Mask Fabrication and Photolithography

Photolithographic masks for all lithography patterns were obtained from Quality Color (Edmonton, Canada) as high resolution film printed on an imagesetter (2540 dpi). Each mask was designed in the CAD program L-Edit. Standard photolithographic techniques were used in forming positive relief photoresist structures on Si wafers as masters for PDMS curing. Briefly, the negative resist SU-8 2050 (Microchem, Massachusetts) was used for the formation of pillar arrays and channel structures. It was spun at 1250 rpm for 60 s to achieve a thickness of 100 μm for pillar arrays and 2000 rpm for 60 s for a thickness of 40 μm for channel structures. Pre-bake was necessary for 2 hrs at 100° C. to remove excess solvent. UV exposure time of 96 s was used, followed by a post bake at 100° C. for 1 hr. Development was achieved using Microchem SU-8 developer for 15 min.

### PDMS Fabrication and Bonding

Upon master fabrication all Si wafers were gas phase silanized, to facilitate easy removal of cured PDMS, with trichloro(1H, 1H, 2H, 2H-perfluorooctyl)silane by placing the wafers and 10 μL of silane, contained in a glass vial, in a vacuum desiccator over night. Polydimethylsiloxane (PDMS) (Sylgard 184, Dow Corning; Midland, Mich.) curing was achieved according to established methods. Briefly, a 10:1, prepolymer cross-linker ratio, by weight, was mixed and placed under vacuum to remove trapped air bubbles. With air bubbles removed the mixed PDMS was poured over the positive relief masters and placed under vacuum to remove any remaining air bubbles. Subsequent curing was achieved at 90° C. for 1 hr. Bonding of the two layer PDMS 96 spot device was achieved using an O₂ plasma to generate surface —OH groups for covalent attachment. The following parameters were used; P=0.200 Torr, O₂=25% forward power=100 W

### Alignment Microscope

A home built alignment microscope was constructed to facilitate alignment of Au patterned slides and microchannel devices. It consists of one x,y,z micron translation stage coupled to a θ stage. PDMS pieces are placed up side down on glass frames which are stationary and positioned within a slot holder. The PDMS is affixed to the glass frame through conformal contact. Au patterned slides are mounted on a holder attached to the translation stages and are free to move. Both pieces are brought close together so that features on both the PDMS and glass slide can be seen at the same focal length, using a 6.3×0.20 NA lens. Alignment can be adjusted and the glass slide moved into contact with the stationary PDMS when satisfied. Upon bonding, a vacuum is applied to the bottom holder and the PDMS is removed from the glass frame, due to its weaker adhesion to the border of the glass frame, as the bottom holder is lowered.

The analytical techniques described herein may be applied while fluid is flowing through one of the microfluidic spotting devices described. The techniques may be applied to detect constituents of the fluid, as for example any biomolecule, such as nucleic acids, proteins, peptides, antibodies, enzymes, and cell wall components, including natural, modified and synthetic forms of the biomolecules. Various methods may be used to bring fluid to the inlet reservoirs, for example through attachment tubing.

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims.

1. A microfluidic spotting device, comprising:
a substrate patterned with an array of spots, the substrate being suitable for use in a surface based analytical method;
a channeled substrate attached to the substrate; and
a channel network formed at least partially in the channeled substrate, the channel network having more than one distinct channel path, each channel path including an inlet channel and an outlet channel and being uniquely associated with and passing across a spot or group of spots.

2. The microfluidic spotting device of claim 1 in which each channel path has a length, the lengths of each channel path being equal and each channel presenting equal resistance to flow through the channels.

3. The microfluidic spotting device of claim 1, wherein at least one spot in a channel is an elongate spot extending along the channel.

4. The microfluidic spotting device of claim 1, wherein at least one spot is formed of contiguous metal passing across multiple channels.

5. The microfluidic spotting device of claim 1, wherein at least one channel has more than one inlet channel.

6. The microfluidic spotting device of claim 2, wherein more than one outlet channel is connected to a common drain.

7. The microfluidic spotting device of claim 2, wherein at least one inlet channel is in communication with a reaction bed upstream from the corresponding spot.

8. The microfluidic spotting device of claim 1, further comprising a top substrate, the top substrate being attached to the second substrate such that the second substrate is an intervening substrate between the top substrate and the first substrate.

9. The microfluidic spotting device of claim 8, wherein the intervening substrate has openings corresponding to the locations of spots on the spotted substrate.

10. The microfluidic spotting device of claim 8, wherein at least one channel is at least partially formed in the top substrate.

11. The microfluidic spotting device of claim 1, wherein the second substrate is attached directly or indirectly to the spotted substrate by an attachment surface, the channel network being formed on the attachment surface of the second substrate.

12. The microfluidic spotting device of claim 1, wherein the array of spots is an array of coinage metal spots.

13. The microfluidic spotting device of claim 1, wherein the channel network comprises channels, and the channels are parallel to the plane of the array of spots.

14. The microfluidic spotting device of claim 1 in which the inlet channels of the channel network are connected to receive fluid from a microtitre plate.

15. The microfluidic spotting device of claim 1 made of material suitable for use in surface Plasmon resonance analysis.

16. A microfluidic spotting device, comprising:
a spotted substrate patterned with an array of spots;
a microtitre plate having wells; and
a channel network between the spotted substrate and the channeled substrate coupling the wells to the array of spots.

17. The microfluidic spotting device of claim 16, wherein the array of spots is a two-dimensional array.

18. The microfluidic spotting device of claim 16, wherein at least one spot is an elongate spot.

19. The microfluidic spotting device of claim 16, wherein the channel network comprises inlet channels and outlet channels, each spot being in communication with a distinct inlet channel.

20. The microfluidic spotting device of claim 19, wherein more than one
outlet channels are connected to a common drain.

21. The microfluidic spotting device of claim 20, wherein each inlet channel corresponding to the common drain has the same length and cross-sectional area.

22. The microfluidic spotting device of claim 16, wherein the channel network comprises reaction beds upstream from the array of spots.

23. The microfluidic spotting device of claim 16, wherein the channel network is formed from a channeled substrate attached to the spotted substrate.

24. The microfluidic spotting device of claim 23, comprising more than one channeled substrate, such that the channel network is a three-dimensional channel network.

25. The microfluidic spotting device of claim 23, wherein the channels are parallel to the array of spots.

26. The microfluidic spotting device of claim 1, wherein the spots are metallic spots.

27.-47. (canceled)

48. A microfluidic spotting device, comprising:
a first substrate patterned with an array of spots;
a second substrate attached to the first substrate; and
a channel network formed between the first substrate and the second substrate, each spot being in fluid communication with a distinct channel path through the channel network.

49. The microfluidic spotting device of claim 48, wherein the channel network comprises channels formed at least partly in at least one of the first substrate and the second substrate, each spot being in communication with an inlet channel leading to the spot and an outlet channel leading away from the spot.

50. The microfluidic spotting device of claim 48 with any one or more of:
the substrate being made of material suitable for surface Plasmon resonance analysis;
each channel path being uniquely associated with and passing across a spot or group of spots;
each channel path has a length, the lengths of the channel path being equal and each channel presenting equal resistance to flow through the channels;
at least one spot in a channel is an elongate spot extending along the channel;
at least one spot is formed of a strip of material passing across multiple channels;
at least one channel has more than one inlet channel;
more than one outlet channel is connected to a common drain;
at least one inlet channel is in communication with a reaction bed upstream from the corresponding spot;
a top substrate being attached to the second substrate such that the second substrate is an intervening substrate between the top substrate and the first substrate;
one or more intervening substrates having openings corresponding to the locations of spots on the spotted substrate;
at least one channel is at least partially formed in a top substrate;
the second substrate is attached directly or indirectly to the spotted substrate by an attachment surface, the channel network being formed on the attachment surface of the second substrate;
the array of spots is an array of coinage metal spots;
the channel network comprises channels, and the channels are parallel to the plane of the array of spots; and
the inlet channels of the channel network are connected to receive fluid from a microtitre plate.

51. (canceled)
